(19) United States
(12) Patent Application Publication
(10) Pub. No.: US 2006/0286555 A1
(43) Pub. Date: Dec. 21, 2006

(54) Microarray support for bioprobe synthesis

(76) Inventors: Marinus Gerardus Johannus Van Beuningen, Oss (NL); Tim Kievits, Vught (NL); Rob Ruijtenbeek, Utrecht (NL)

Correspondence Address: AMSTER, ROTHSTEIN & EBENSTEIN LLP, 90 PARK AVENUE, NEW YORK, NY, 10016, US

(21) Appl. No.: 10/568,360

(22) PCT Filed: Aug. 20, 2004
(86) PCT No.: PCT/EP04/09351

§ 371 Date: Feb. 13, 2006

(30) Foreign Application Priority Data
Aug. 21, 2003 (EP) 03447216.7

(51) Int. Cl. C12Q 1/68; G01N 33/53; C12M 1/34; G01N 33/00

(52) U.S. Cl. 435/6; 435/7.1; 435/287.2; 977/924; 436/94

(57) ABSTRACT

A system for conducting chemical and/or biochemical reactions is provided, comprising a reaction substrate (A) with a multiplicity of reactor zones (C), said reaction substrate (A) having a substantially flat top surface and a substantially flat bottom surface, the reaction substrate (A) being porous with a multiplicity of essentially parallel pores enabling liquid flow through, said pores having a diameter of about 10 μm to 10 nm, characterised in that said flat top surface is bonded to the bottom of a first rigid support (B), said rigid support (B) comprising a multiplicity of through going holes extending from the top of said rigid support (B) to the bottom of said rigid support (B), and said through going holes defining the reactor zones (C). The invention further relates to the use of said system, an apparatus comprising said system as well as an incubation device for holding said system and a loading station.

### FIELD OF THE INVENTION

A system for conducting chemical and/or biochemical reactions is provided, comprising a reaction substrate with a multiplicity of reactor zones, said reaction substrate having a substantially flat top surface and a substantially flat bottom surface, the reaction substrate being porous with a multiplicity of essentially parallel pores enabling liquid flow through, said pores having a diameter of about 10 μm to 10 nm, characterised in that said flat top surface is bonded to the bottom of a first rigid support, said rigid support comprising a multiplicity of through going holes extending from the top of said rigid support to the bottom of said rigid support, and said through going holes defining the reactor zones. The invention further relates to the use of said system, and an apparatus comprising said system as well as an incubation device for holding said system and a loading station.

### BACKGROUND OF THE INVENTION

Current oligonucleotide and peptide synthesis systems use a particle-based technology since the late 1980s. These systems use Controlled Pore Glass (CPG) or polymer resin solid phase in a column. The synthesis takes place in a step-by-step process in which each of the nucleotides or amino acids building blocks are added.

A fluid delivery station known as an oligonucleotide or peptide synthesiser controls the reagents and building blocks needed for the synthesis. These systems are equipped with a number of vials for the storage of reagents and building blocks needed in the process. Typically, 6-10 vials for DNA synthesis and 25 vials for peptide synthesis are required. The throughput of these systems is relatively slow because of the time needed for completion of chemical reaction steps and because of the stringent washing, which is needed to clean the column and the tubing.

The current explosion of genomic information has resulted in an enormous boom in small scale DNA synthesis. The use of PCR and oligonucleotide microarrays has resulted in a need for low cost DNA synthesis on a scale as small as e.g. 0.05-0.1 nmol. Current CPG based synthesis is scalable from a 200-nmol synthesis to a mmol scale for large scale synthesis, which is used in antisense technologies. However, CPG based synthesis has problems in delivering at a high throughput basis small amounts of oligonucleotides. Furthermore, the quality of the resulting oligonucleotides is often poor. In this regard, optical quality control is not possible with a CPG based approach.

Therefore, there is a need for systems enabling low-cost and small-scale chemical and/or biochemical reactions for synthesising polymers.

### SUMMARY OF THE INVENTION

In order to provide low-cost, small-scale synthesis the present invention provides a system for conducting chemical and/or biochemical reactions, comprising a reaction substrate with a multiplicity of reactor zones, said reaction substrate having a substantially flat top surface and a substantially flat bottom surface, the reaction substrate being porous with a multiplicity of essentially parallel pores enabling liquid flow through, said pores having a diameter of about 10 μm to 10 nm, about 5 μm to 25 nm, about 1 μm to 50 nm, 0.5 μm to 0.1 μm, or about 0.2 μm, characterised in that said flat top surface is bonded to the bottom of a first rigid support, said rigid support comprising a multiplicity of through going holes extending from the top of said rigid support to the bottom of said rigid support, and said through going holes defining the reactor zones.

### DETAILED DESCRIPTION

The present invention relates to a system for the high-throughput, multiparallel and fast synthesis of high quality polymers, such as oligonucleotides and peptides, at low cost and with minimal reagent consumption and on-line coupling efficiency monitoring (e.g. Trityl or Fmoc monitoring). The invention further relates to the use of said system, and an apparatus comprising said system as well as an incubation device for holding said system and a loading station.

In the present specification and the appended claims, the singular forms “a”, “an”, and “the” include plural references unless the context clearly dictates otherwise. Unless defined otherwise, all technical and scientific terms used herein have the same meaning as commonly understood to one of ordinary skill in the art.

Substrate

In view of conducting chemical and/or biochemical reactions at low-cost, at small-scale and on-line coupling efficiency monitoring, the choice of the reaction substrate is of prime importance. The present invention provides a porous, three-dimensional (3D) structure. The three dimensional structure of the reaction substrate enlarges the reaction surface, compared to two-dimensional (2D) substrates. For instance, the 3D structure of commercially available aluminium oxide, such as Anopore™ (Whatman), enlarges the reaction surface by 500 compared to a non-porous two-dimensional substrate. This surface enlargement is sufficient for small-scale synthesis in the sub-nanomolar scale. The porosity of the reaction substrate may result from a multiplicity of essentially parallel pores, the pores being perpendicular to the upper and lower services of the substrate. An advantage of these pores is that they enable effective liquid flow-through. This liquid flow-through provides efficient and fast washing and fluid addition. Furthermore, this liquid flow-through enables highly efficient reaction kinetics and washing efficiencies, resulting in high quality reaction products, because diffusion distances, which are rate limiting in other solid phase support materials (e.g. resins, gels, CPG and the like), are extremely short, and reaction components which have not been used are immediately and efficiently removed from the area of synthesis and can thus not interfere with new reactions.

The material of the porous substrate may be, for example, a metal, a ceramic metal oxide or an organic polymer. In view of chemical resistance, strength, rigidity and optical transparency, a metal or a ceramic metal oxide may be used. Above all, in view of heat resistance, a metal oxide may be used. Exemplary suitable materials for use as substrates in the present invention are metal oxides. Metal oxides provide a substrate having both a high channel (pore) density and a high porosity, allowing reaction substrates comprising a high amount of first binding substances per (2D) surface unit. In addition, metal oxides are highly transparent for visible light. Metal oxides are relatively cheap substrates that do not require the use of any typical microfabrication technology. Metal oxide membranes having a multiplicity of essentially parallel pores can be manufactured through electrochemical etching of a metal sheet. Metal oxides considered are, among others, oxides of zirconium, silicium, mullite, cordierite, titanium, zeolite or zeolite analog, tantalum, and aluminium, as well as alloys of two or more metal oxides and doped metal oxides and alloys containing metal oxides. The kind of metal oxide is not especially limited. As a metal, for example, a porous substrate of stainless steel (sintered metal) can be used. For applications not requiring heat resistance, a porous substrate of an organic polymer can also be used if it is sufficiently mechanically stable. Such membranes have a multiplicity of essentially parallel pores with well controlled diameter and useful chemical surface properties. The diameter of the pores is an important factor in determining the total amount of molecules anchored per the total dimensions of the reaction substrate. In the present invention, the pores have a diameter of about 10 μm to 10 nm, about 5 μm to 25 nm, about 1 μm to 50 nm, 0.5 μm to 0.1 μm, or about 0.2 μm. A substrate as useful in the method according to the present invention may comprise for instance about 10⁷ pores per mm². These dimensions are not to be construed as limiting the present invention. The small pore diameter in this set up ensures fast reaction times, such as for phosphoramidite and Fast-Fmoc chemistry, because of the small diffusional distances. In addition, the porous substrate provides minimal dead volumes. Patent applications EP-A-0 975 427 and WO 99/02266, which discloses the use of Anopore™ as a porous substrate, are exemplary in the above respect, and are specifically incorporated in the present invention. This Anopore™ substrate has interconnected pores of about 0.2 μm in diameter which extend from the first top surface to a defined region within the substrate, followed by additional pores or channels of significantly less diameter, e.g. about 0.02 μm, which extend thereafter to the bottom of the surface. According to the above, the present invention provides a reaction substrate comprising a substantially flat top surface and a substantially flat bottom surface, the reaction substrate being porous with a multiplicity of essentially parallel pores enabling liquid flow through. It will further be understood that the term “essentially parallel pores” includes through-going oriented channels, is not restricted to discrete channels, but also includes branched pores which are connected to adjacent pores in the substrate.

The reaction substrate of the present invention may be made of an organic or inorganic material, including glass or a metal oxide, such as, for example, aluminium oxide.

The metal oxide membranes are optically transparent, especially if wet, which allows for assays using various optical techniques. The transparency allows a real time measurement or continuous process monitoring to be made, e.g. for quality control, using a CCD (charged coupled optical detector) camera, low angle laser scanner or other, suitable optical scanner. The optical transparent substrate allows colorimetric based detection, which may be used to monitor reagent addition as well as to monitor coupling efficiencies (e.g. Trityl-monitoring, Fmoc-monitoring), monitor reaction rates and reaction products. In a further embodiment, the use of a system for monitoring the chemical and/or biochemical processes is provided.

Accordingly, the present invention provides a system, wherein the reaction substrate is optically transparent or translucent

Accordingly, the present invention provides a system for conducting chemical and/or biochemical reactions as described herein, wherein said chemical and/or biochemical reactions are monitored optically, possibly in a continuous fashion.

Monitoring of the reaction processes within the substrate is by signals generated by said reactions. Said signals may be transient, i.e. passing especially quickly into and out of existence. The term “transient signal” as used in the present invention refers to e.g. a pulse. In the present invention, each sequential addition of a second reaction component may be monitored for the generation of a signal pulse. The level and intensity of said pulse may be directly linked to the reaction resulting upon addition of said second reaction component. The signal detection may be a quantitative or a qualitative observation or both. The system of the present invention provides a signal via optical radiation. As used herein, the term “optical radiation” can include radiation that can be used to transmit signals, such as radiation in the visible, ultraviolet, infrared and/or other portions of the electromagnetic radiation spectrum. It will be understood that reactions performed according to the present invention may be monitored by for example bioluminescence, radioactive radiation, chemi-luminescence and/or fluorescence.

Detection methods of the generated signals are well known in the art. Signals may be detected or visualized in a variety of ways, with the particular manner of detection being chosen based on the reporter system which is utilized. Representative detection means include scintillation counting, auto-radiography, optical detection such as fluorescence measurement, colorimetric measurement, light scattering, and the like.

Chemical and/or Biochemical Reactions

A wide diversity of different polymers can be synthesised in the present substrate, and consequently a vast array of chemical and/or biochemical reactions can be conducted using the present substrate.

Accordingly, the present invention provides a system for conducting chemical and/or biochemical reactions as described herein, wherein said chemical and/or biochemical reactions are polymer synthesis reactions, including nucleotide synthesis reactions, peptide synthesis reactions, and sugar polymer synthesis reactions.

The reaction substrate, i.e. the walls of the pores, may be prepared by coating or chemically modifying the reaction substrate with a layer of a substance having reactable functional groups such as amino groups. In an embodiment, an aminosilane such as 3-amino propyltrimethoxysilane (APTS) or N-(2-aminoethyl-3-aminopropyl)timethoxysilane (EDA) is used, but other reactable substances may be used. Following this first preparation step, due to the presence of the reactable functional groups, the entire surface of the reaction substrate is hydrophilic. In an embodiment, the reactor zones contain reactable functional groups such as, but not limited to, amino hydroxyl, sulfhydryl, carboxyl, amine, epoxy, aldehyde, ketone, vinylsulfonyl or haloalkyl groups.

Typically, a first molecule, or first building block, is then anchored to the walls of the pores of the substrate as a starting material for further chemical and/or biochemical reactions. This first building block is subsequently extended by covalently coupling a next or further building block, which can be similar to or different from the first building block. A building block may be a chemical moiety, a chemical building group, a chemical monomer, a polymer or any other appropriate molecule, such as, for example nucleotides, nucleosides, sugars, or amino acids. It will be understood that the term “building block” connotes dimers, tri-mers, oligomers etc. of the nucleotides, nucleosides, amino acids and sugars, such as, for example, polynucleotides containing a sequence of, for example, 2, 3, 4, 5, 6, 7, 8 or 9 nucleotides, peptides containing a sequence of, for example, 2, 3, 4, 5, 6, 7, 8 or 9 amino acids.

The expression “first building blocks” as used in the present invention refers to one of the reaction components which is present within predefined regions on the substrate. A first building block may be attached to said substrate, e.g. covalent, non-covalent or absorptive.

The present invention relates to a process to synthesise polymers, characterised in that said polymers are attached to the pores of a system as described herein.

A variety of techniques have been described for synthesizing and/or immobilizing arrays of poly-nucleotides, including in situ synthesis, where the polynucleotides are synthesized directly on the surface of the substrate (see, e.g., U.S. Pat. No. 5,744,305 to Fodor, et al.,) and attachment of pre-synthesized polynucleotides to the surface of a substrate at discrete locations (see, e.g., WO 98/31836). Additional methods are described in WO 98/31836 at pages 41-45 and 47-48, among other places. Similarly, a variety of techniques have been described for synthesizing and/or immobilising arrays of (poly)peptides. The present invention relates to the techniques commonly known in the art, such as, for example, Boc-chemistry, Fmoc-chemistry (see for instance Atherton and Sheppard in “Solid phase peptide chemistry, a practical approach” IRL press at Oxford University Press, UK, 1989), Fastmoc chemistry, Spot™ technology (Jerini AG, Berlin Germany) and Photochemical methods. All these 10 patents and patent applications are specifically incorporated herein by reference.

The terms “covalent” and “non-covalent” bond are further explained in the following examples. For instance, for DNA synthesis, the pores may be pre-coated with poly-L-lysine. This forms a stable but an essentially non-covalent bond between the nucleotide and the substrate surface. A particular suitable example of covalent immobilization comprises a silanation step as well known in the art. Reference is for instance made to the technology described in WO 01/12846 in this respect.

The present invention is suitable for use with any of these currently available, or later developed, techniques. Moreover, while the in situ synthesis method is described utilizing phosphoramidite reagents, it will be recognized that other reagents utilizing other synthesis strategies can also be employed, and in certain circumstances may be preferable, depending on the stability of the reaction substrate to the synthesis conditions. Non-limiting examples of suitable chemistries and reagents are described, for example in Oligonucleotide Synthesis: A Practical Approach, M. J. Gait, Ed., IRL Press, Oxford, England, 1985. While the backbones of the polynucleotides will typically be composed entirely of “native” phosphodiester linkages, they may contain one or more modified linkages, such as one or more phosphorothioate, phosphoramidite or other modified linkages. As a specific example, one or more immobilized polynucleotides may be a peptide nucleic acid (PNA), which contains amide interlinkages. Additional examples of modified bases and backbones that can be used in conjunction with the invention, as well as methods for their synthesis can be found in, for example, Uhlman & Peyman, 1990, Chemical Review 90(4):544-584; Goodchild, 1990, Bioconjugate Chem. 1(3):165-186; Egholm et al., 1992, J. Am. Chem. Soc. 114:1895-1897; Gryaznov et al., J. Am. Chem. Soc. 116:3143-3144, in www.active_motif.com, Efimov et al., 1998, Nucleic Acids Research 26:566-575 and Efimov et al., 1999, Nucleic Acids Research 27:4416-4426 for gripNAs™, in www.exigon.com for LNA™ (Exiqon, Vedbaek, Denmark), as well as in the references cited in all of the above.

Reaction components may be added to initiate a chemical and/or biochemical reaction. According to the processes encompassed by the present invention, a single second reaction component may be added but additional reaction components may be included as well. As such, a second reaction component may be a reagent mix comprising enzymes and/or substrates or any mixture thereof. The system according to the present invention allows the sequential addition of at least one second reaction component. At each subsequent addition, the composition of said at least one second reaction component may be changed.

Reactor Zones

In the present invention, the chemical and/or biochemical reaction or reactions are conducted at a spatially defined region in the substrate, i.e. in particular pores. A spatially defined region presents a reactor zone, in which a chemical and/or biochemical reaction may be conducted. The substrate may contain various, i.e. more than one, spatially defined regions. Each spatially defined region is individually addressable for selectively delivering particular liquid reaction components, such as, for example, solvents, reagents, wash solutions, enzymes and/or building blocks. The spatially defined regions are such that liquid movement between adjacent reactor zones is prevented. The reactor zones form a pattern, for instance, an array of reactor zones arranged in rows and columns. The reactor zones may be rectangular, but any suitable discrete form may be used, such as, but not limited to, circular or triangular forms.

In an embodiment, the substantially flat top surface of the reaction substrate is bonded to the bottom of a first rigid support, said rigid support comprising a multiplicity of through going holes extending from the top of said rigid support to the bottom of said rigid support, and said through going holes defining the reactor zones. The rigid support may have, for instance, cavities of about 3.5×3.5 mm with a height of approximately 3 mm, which creates a holding reaction cavity of up to 30 μl. Hence, the holding reaction cavity or “hole” may, possibly reversibly, contain reaction components, such as, for instance, a solvent, reagent, wash solution, enzyme, monomer and/or building blocks used for the polymer synthesis reaction. The dimensions of the rigid support may be based on the SBS (Society for Biomolecular Screening) footprint, and may consist of a plastic, rigid, mechanically stable, chemical and/or temperature resistant support to which the reaction substrate, e.g. a porous aluminium oxide sheet is bonded via heat, laser welding, ultrasonic welding, chemical bonding, glueing, moulding or any other suitable method. An additional advantage of the reaction substrate being bonded to the rigid support is that the rigid support strengthens and supports the substrate, which allows easier handling of the substrate.

The first rigid support is made of a suitable plastic material, e.g. LCP, Topas® or polypropylene, but it can also be made out of other suitable materials such as glass or silicon.

The material used must be chemically resistant and/or heat resistant up to 120° C., robot compatible, and/or optically compatible, e.g. flat and with minimal autofluorescence or auto-absorbance. Further, the material should have minimal a specific binding properties for the components used in the chemical and/or biochemical reactions. The rigid support may be black to minimize autofluorescence, auto-absorbance and refractive back scattering of light. Alternatively, it is possible to provide the first rigid support with a coating to obtain the desired non-reflective properties. The rigid support is attached to the reaction substrate by moulding, glueing, chemical bonding, thermal bonding, such as for instance via heat, laser welding, or ultrasonic welding, or any other suitable method known in the art.

Accordingly, the present invention provides a system for conducting chemical and/or biochemical reactions, comprising a reaction substrate with a multiplicity of reactor zones, said reaction substrate having a substantially flat top surface and a substantially flat bottom surface, the reaction substrate being porous with a multiplicity of essentially parallel pores enabling liquid flow through, said pores having a diameter of about 10 μm to 10 nm, about 5 μm to 25 nm, about 1 μm to 50 nm, 0.5 μm to 0.1 μm, or about 0.2 μm, characterised in that said flat top surface is bonded to the bottom of a first rigid support, said rigid support comprising a multiplicity of through going holes extending from the top of said rigid support to the bottom of said rigid support, and said through going holes defining the reactor zones. It will be clear that the pores and through going holes are essentially coaxial.

It will be appreciated that the present invention connotes the use of the system as described herein for conducting chemical and/or biochemical reactions, including the synthesis of polymers, wherein said polymers may be, for instance, oligonucleotides, peptides or sugar chains. Similarly, the use of the system as described herein is indicated for synthesising in parallel different polymers. In each reactor zone of the system, a different or the same polymer may be synthesised. The term “different polymers” does not exclude that different polymers of the same group, e.g. the group of oligonucleotides, the group of peptides or the group of sugar chains, are synthesised. In other words, all the polymers synthesised in parallel on the system of the present invention, may all be of the same group, e.g. the group of oligonucleotides, the group of peptides or the group of sugar chains, but the particular polymer, for instance a first oligonucleotide with a specific sequence, differs from other polymers of the same group, for instance, a second oligonucleotide with a sequence differing from the sequence of the first oligonucleotide. Also, all the polymers synthesised in parallel on the system of the present invention may be of different groups, e.g. peptides and PNA in the same synthesis.

Further, the present invention provides a system as described herein, wherein the bottom surface of said reaction substrate is bonded to the top of a second rigid support, said second rigid support having through going holes extending from the top of the second rigid support to the bottom of the second rigid support, and said holes of the second rigid support are aligned with the holes of the first rigid support.

In an embodiment, part of the essentially parallel pores are closed by masking, preventing liquid flow through. The masked pores may border the reactor zones.

Accordingly, the present invention provides a system for conducting chemical and/or biochemical reactions, comprising a reaction substrate with a multiplicity of reactor zones, said reaction substrate having a substantially flat top surface and a substantially flat bottom surface, the reaction substrate being porous with a multiplicity of essentially parallel pores enabling liquid flow through, said pores having a diameter of about 10 μm to 10 nm, about 5 μm to 25 nm, about 1 μm to 50 nm, 0.5 μm to 0.1 μm, or about 0.2 μm, characterised in that part of said pores are masked, the masked pores may define the outer borders of the reactor zones.

Accordingly, the present invention further provides a system for conducting chemical and/or biochemical reactions, comprising a reaction substrate with a multiplicity of reactor zones, said reaction substrate having a substantially flat top surface and a substantially flat bottom surface, the reaction substrate being porous with a multiplicity of essentially parallel pores enabling liquid flow through, said pores having a diameter of about 10 μm to 10 nm, characterised in that (i) part of said pores are masked, by filling said pores with a masking polymer the masked pores defining the outer borders of the reactor zones; and (ii) said flat top surface is bonded to the bottom of a first rigid support comprising a multiplicity of through going holes extending from the top of said rigid support to the bottom of said rigid support, and said through going holes defining the reactor zones.

Another elaboration of defining spatially addressable regions optically creates an array of reactor zones. A reaction substrate is washed with an organosilane that chemisorbs to the reaction substrate, e.g. glass, to coat the reaction substrate. The organosilane coating is irradiated by deep UV light through an optical mask that defines a pattern of an array. The irradiation cleaves the Si—C bond to form a reactive Si radical. Reaction with water causes the Si radicals to form polar silanol groups. The polar silanol groups constitute spots on the array and are further modified to couple other reactable molecules to the spots, as disclosed in U.S. Pat. No. 5,324,591, incorporated by reference herein. For example, a silane containing a biologically functional group such as a free amino moiety can be reacted with the silanol groups. The free amino groups can then be used as sites of covalent attachment for further building blocks.

For example, in photochemical resist-photolithograpy (Mrksich and Whitesides, Ann. Rev. Biophys. Biomol. Struct. 25:55-78, 1996), a reaction substrate, e.g. a glass plate, is uniformly coated with a photoresist, and a photo mask is placed over the photoresist coating to define the “array” or pattern desired. Upon exposure to light, the photoresist in the unmasked areas is removed. The entire photolithographically defined surface is uniformly coated with a hydrophobic substance such as an organosilane that binds both to the areas of exposed reaction substrate and the areas covered with the photoresist. The photoresist is then stripped from the reaction substrate surface, exposing an array of spots of exposed reaction substrate. The reaction substrate then is washed with an organosilane having terminal hydrophilic groups or chemically reactable groups such as amino groups. The hydrophobic organosilane binds to the spots of exposed reaction substrate with the resulting reaction substrate having an array of hydrophilic or reactable spots (located in the areas of the original photoresist) across a hydrophobic surface. The array of spots of hydrophilic groups provides a substrate for binding.

In this manner a system is obtained, which can be made according to SBS standard formats allowing the use of standard screening instrumentation, especially in automated robotic platforms. Using for example a microplate format with an array of 96, 384 or 1536 reactor zones or wells allows a parallel processing of a large number of chemical and/or biochemical reactions resulting in a very efficient high throughput synthesis. The microplate of the system may be bar-coded for identification.

Accordingly, the present invention provides a system as described herein, wherein said rigid support has 96, 384 or 1536 holes and/or reactor zones.

Masking

The essentially parallel pores of the reaction substrate may be masked, which results in closure of these pores. The masking process is done by means of a masking polymer, which enables filling of the pores and which can subsequently be activated by light (photo mask) or chemicals using a mask, which specifically close certain areas on the substrate. Examples of such a masking polymers are polyacrylamide, photocrosslinkable polymers, photoreactive glues etc, all of which are well known in the art. Further, the closure of the pores may be reversible, i.e. after initial closure, the pores may be re-opened. This process of closure and opening may be re-iterated, according to the needs of the user. After hardening the masking polymer for closure, the pores may thereafter be cured by removing the masking polymer.

Accordingly, the present invention provides a system according to the present invention, wherein the masking is carried out by a masking polymer, including polyacrylamide, photocrosslinkable masking polymers and photoreactive glues.

Accordingly, the present invention provides a system according to the present invention, wherein a subset of said pores are, possibly reversibly, masked.

System

The system of the present invention is possibly adapted to an SBS (Society for Biomolecular Screening) standard format, allowing the use of standard screening instrumentation, for instance on existing robotic HTS (high throughput screening) platforms. Adaptation to standard instrumentation formats allows easy fluid transfer, such as for instance by pipettor or a fast multidrop system (e.g. reagent addition every minute). Using for example a system with an array of 96, 384 or 1536 reactor zones according to SBS standards allows parallel synthesis of a large number of polymers, resulting in a very cost-efficient price per polymer.

In a further embodiment, a system is provided wherein the reaction substrate is held in a plane by a holding means.

The cavities of the first rigid support, as well as reactor zones, have well defined dimensions.

An amount of reaction component is added to one or more of the reactor zones of the system, the amount of added reaction components being calculated on the basis of the dimensions of the reactor zone, including the possible presence of the cavity of the first rigid support. A flow is generated through the reaction substrate in the pores of the reactor zones whereby the liquid volume of the reaction components is forced to pass through the pores in the substrate from the upper side of the substrate to the lower side of the substrate, under conditions that are favourable to conduct a chemical and/or biochemical reaction, which may include a reaction with the walls of the pores and/or building blocks. Any signal generated in any of the reactor zones may be read, and from said signals the presence, amount, stage and/or identity of said chemical and/or biochemical reactions may be determined. The system may contain a cover, which may be used for sealing the reactor zones. When the system is covered by a transparent material, such as a glass or plastic cover, the reactor zones can be analysed and the reading signal can be determined through the cover. The cover may be provided with a heating element, for example, by incorporating transparent electrical wires in the cover material. The cover can be heated in this manner to prevent condensation during conduction of the reactions.

The system as described herein may comprise a reaction manifold for selectively delivering particular reaction components, such as, for instance, solvents, reagents, wash solutions, enzymes and/or monomers to said reaction zones.

The porous nature of the substrate facilitates the pressurized movement of fluid, e.g. the sample solution, through its structure. In contrast to two-dimensional substrates, the flow-through nature of a 3-dimensional substrate or microarray, as employed in the methods as described herein, gives significantly reduced reaction times and results in increased purity.

A positive or negative pressure may be applied to the system of the present invention in order to pump the reaction solution dynamically up or down through the substrate pores. Alternatively, the substrates may be subjected to a centrifugal force. The duration, type and strength of the force applied will determine the level of displacement of reaction components within the substrate. An advantage of the present invention is that reaction components may be displaced or refreshed by simple flowing off.

In one embodiment of the present invention, removal of analyte and/or reaction components is by a means selected from the group comprising centrifugal force, pressure, and suction including vacuum.

The flow of the reagents through the reaction substrate may be achieved by a vacuum manifold positioned below the reaction substrate or by the use of a centrifuge based system. In addition, the present invention provides a system with means for applying a pressure. Also, the system provides a means for inducing a reversible flow through the reaction substrate. Accordingly, the system presents efficient and fast washing and fluid addition.

In another aspect, the invention provides an apparatus for conducting chemical and/or biochemical reactions comprising a system as described above, an incubation device for holding said system, a loading station, possibly a dispensing and aspiration station, possibly a pressure or vacuum application station, and possibly a reading station.

The invention also provides an incubation device to be used in the system of the invention. Also, the invention provides an apparatus for conducting high throughput synthesis, comprising a system of the invention, a device for linearly moving the incubation device along a plurality of stations including a station for loading a the system into the incubation device, a station for dispensing a liquid into the holes or on the reactor zones of the microplate, and a reading station for individually illuminating each substrate of the system, wherein a device is provided for moving the incubation device with the microplate with respect to the reading station in mutually perpendicular directions.

The present invention relates further to a system for conducting chemical and/or biochemical reactions, comprising a reaction substrate with a number of reaction zones, and an incubation device for holding the reaction substrate.

### SHORT DESCRIPTION OF THE FIGURES

FIG. 1 shows a top SBS view of an embodiment of the system of the invention. A: substrate; B: plastic SBS footprint; C: Reaction zone cavity.

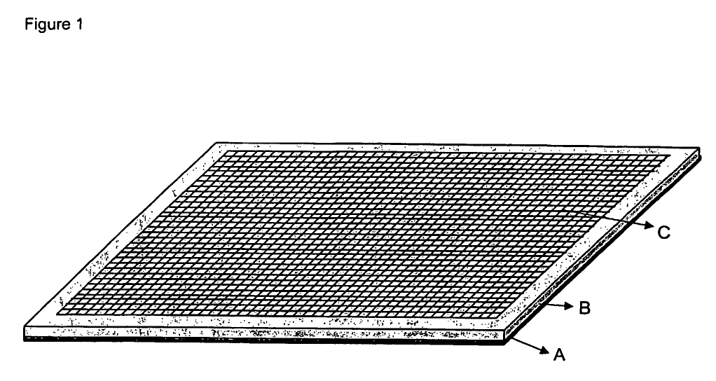

FIG. 2 is an exploded view of part of the system of FIG. 1. A: substrate; B: plastic SBS footprint; C: Reaction zone cavity.

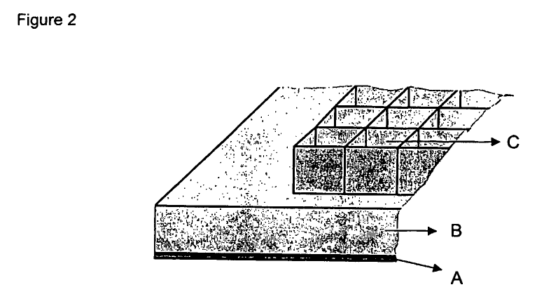

1. System for conducting chemical and/or biochemical reactions, comprising a reaction substrate with a multiplicity of reactor zones, said reaction substrate having a substantially flat top surface and a substantially flat bottom surface, the reaction substrate being porous with a multiplicity of essentially parallel pores enabling liquid flow through, said pores having a diameter of about 10 μm to 10 nm, characterized in that said flat top surface is bonded to the bottom of a first rigid support, said rigid support comprising a multiplicity of through going holes extending from the top of said rigid support to the bottom of said rigid support, and said through going holes defining the reactor zones.

2. System according to claim 1, wherein a subset of said holes are reversibly, masked.

3. System for conducting chemical and/or biochemical reactions, comprising a reaction substrate with a multiplicity of reactor zones, said reaction substrate having a substantially flat top surface and a substantially flat bottom surface, the reaction substrate being porous with a multiplicity of essentially parallel pores enabling liquid flow through, said pores having a diameter of about 10 μm to 10 nm, characterized in that part of said pores are masked by filling said pores with a masking polymer, the masked pores defining the outer borders of the reactor zones.

4. System according to claim 1, characterized in that part of said pores are masked, by filling said pores with a masking polymer, the masked pores defining the outer borders of the reactor zones.

5. System according to claim 1, wherein chemical moieties, chemical building groups, chemical monomers, molecules or polymers are anchored to the walls of said pores.

6. System according to claim 3, wherein said masking is carried out by a masking polymer, photocrosslinkable masking polymers and photoreactive glues.

7. System according to claim 1, wherein said chemical and/or biochemical reactions are monitored optically.

8. System according to claim 1, wherein said chemical and/or biochemical reactions are polymer synthesis reactions, nucleotide synthesis reactions, peptide synthesis reactions, or sugar polymer synthesis reactions.

9. System according to claim 1, wherein said reaction substrate is made of an organic or inorganic material.

10. System according to claim 1, wherein said reaction substrate is optically transparent or translucent.

11. System according to claim 1 wherein said rigid support is bonded to the reaction substrate by moulding, gluing, thermal bonding, laser welding, or chemical bonding.

12. System according to claim 1, wherein, said rigid support is a chemical and/or temperature resistant material.

13. System according to claim 1, wherein said holes of said rigid support reversibly contain reaction components.

14. System according to claim 1, wherein said holes contain a solvent, reagent, wash solution, enzymes or monomer used for the polymer synthesis reaction.

15. System according to claim 1, comprising a means for applying a pressure.

16. System according to claim 1, comprising a means for inducing a reversible flow through said reaction substrate.

17. System according to claim 1, comprising a reaction manifold for selectively delivering particular solvents, reagents, wash solutions, enzymes and/or monomers to said reaction zones.

18. System according to claim 1, wherein said rigid support has at least 96, 384 or 1536 holes or reactor zones.

19. System according to claim 1, wherein the bottom surface of said reaction substrate is bonded to the top of a second rigid support, said second rigid support having through going holes extending from the top of the second rigid support to the bottom of the second rigid support, and said holes of the second rigid support are aligned with the holes of the first rigid support.

20. System according to claim 1 which is used for the synthesis of polymers.

21. System according to claim 5, wherein said polymers are oligonucleotides, peptides or sugar chains.

22. System according to claim 1, which is used for synthesising synthesizing in parallel different polymers.

23. Process to synthesize polymers, characterized in that said polymers are attached to the pores of a system according to claim 1.

24. Apparatus for conducting chemical and/or biochemical reactions comprising a system according to claim 1, an incubation device for holding said system, a loading station, optionally a dispensing and aspiration station, optionally a pressure or vacuum application station, and optionally a reading station.

25. System according to claim 9, wherein said reaction substrate is made of aluminium oxide.
